(19) United States
(12) Patent Application Publication
(10) Pub. No.: US 2026/0069332 A1
(43) Pub. Date: Mar. 12, 2026

(54) VARIABLE ANGLE LOADING THREADS

(71) Applicants: Wesley Alsup, Eads, TN (US); Michael Tucker, Memphis, TN (US); Michael Beebe, Germantown, TN (US); Christopher Cosgrove, Memphis, TN (US); Eric Pace, Olive Branch, MS (US)

(72) Inventors: Wesley Alsup, Eads, TN (US); Michael Tucker, Memphis, TN (US); Michael Beebe, Germantown, TN (US); Christopher Cosgrove, Memphis, TN (US); Eric Pace, Olive Branch, MS (US)

(21) Appl. No.: 18/829,198

(22) Filed: Sep. 9, 2024

(51) Int. Cl. A61B 17/86

(52) U.S. Cl. CPC A61B 17/8625; A61B 17/864

(57) ABSTRACT

Disclosed herein is a unique Variable Angle Loading Thread that can be utilized on a cannulated or non-cannulated screw, wire, or pin for a variety of end-uses, such as surgical purposes fastening fractured bones together and/or for attaching bone tissue to surrounding tissue. More particularly, this disclosure relates to an industrial and/or surgical medical thread form having a highly angled and extended thread configuration for increasing the pullout strength of the screw as well as torque resistance thereof. For surgical activities, as an example, such characteristics provide effective strength and torque resistance before bone stripping upon implantation. Whether provided with long or short thread (partial or full threading, in other words), such a unique angled and extended thread provides effective surgical fixation fastening fractured bones together and/or for attaching bone tissue to surrounding tissue and/or increasing healing capabilities.

### CROSS-REFERENCE TO RELATED APPLICATION

This application claims priority to U.S. Provisional Ser. No. 65/537,481 , filed on Sep. 8, 2023. The entirety of the provisional application is herein incorporated by reference.

### FIELD OF THE DISCLOSURE

This disclosure generally relates to a cannulated or non-cannulated screw, K-wire, Pins, or similar fixation devices for a variety of end-uses, such as surgical purposes fastening fractured bones together and/or for attaching bone tissue to surrounding tissue. More particularly, this disclosure relates to an industrial and/or surgical medical screw having a highly angled and extended thread configuration for increasing the pullout strength of the screw as well as torque resistance thereof. For surgical activities, as an example, such characteristics provide effective strength and torque resistance before bone stripping upon implantation. Whether provided with long or short thread (partial or full threading, in other words), such a unique angled and extended thread provides effective connection utility and/or healing capabilities. The manufacturing particulars are encompassed within this disclosure, as well, due to the difficulties associated with such a unique screw configuration.

### BACKGROUND OF THE PRIOR ART

Medical screws, wires, pins, and other fixation devices are commonly used for a variety of surgical procedures, including attaching fractured bones and fragments thereof (to aid in such a healing process), anchoring sutures, and attaching sutures to bones to retain soft tissue (such as tendons or ligaments) that may be torn or detached from a target bone. Such medical screws may also fasten and attach prosthetics (plates, for example) to bone. Traditionally, the multiple threads of such medical screws improve surgical repair by increasing the resistance of such a screw from being pulled out from a target bone (or other structure). Such a screw configuration also facilitates actual introduction and implantation within a target bone.

There have been a number of different screw configurations utilized for such a purpose. Conventional screws used for fracture fixation in orthopedic surgery typically and traditionally rely on historic buttress or similar thread designs. Such a traditional thread form of this type provides a certain degree of unidirectional axial loading force resistance but suffers from a lack of adequate multiaxial resistance capabilities. Additionally, such typical fixation technology has suffered in terms of propensity of screw interface stripping which may cause undesirable microfracturing of the target bone which may lead to, as one example, post-surgical failure of fixated bone.

Standard and typical osteopathic (bone) screws utilize standard helical thread configurations, including “high-low” configurations wherein threads exhibit two different (and repeating, in some instances) helical threads of different heights interleaved with each other. Such “high-low” screws seem to exhibit a propensity to counter-rotate after implantation, becoming susceptible to loosening and potential disengagement.

Other types of thread form configurations have been developed in attempts to overcome such typical deficiencies with some success. Interlocking thread configurations have been developed, as well, again to a certain level of improvement, but with the need for more complex geometry dimensions overall. Also of note are curved thread form configurations that turn upward in a spherical arced shape within the helical portion thereof. Such structures provide some degree of support, certainly, but the curved base of such extended threads is lacking a certain level of support due to the curvature of the threads themselves. In addition to curves, rectangles, triangle, v-shaped, chevron, or square threads have all been tried or tested with varying degrees of success.

Although these types of improvements allow for some increases in distributive forces within a fracture site, there still remain significant limitations as to the propensity for such screws to accord necessary strength to avoid stripping and loosening from target bones over time. Such situations can, as noted above, lead to microfractures within target bone that would hinder healing rather than help.

Such thread deficiencies as noted above are noticeable within industrial screws, as well, whether agricultural, aerospace, or even construction types (among many others). The need for improvements analogous to such medical concerns are likewise important within industrial areas, too.

A medical or industrial thread form is thus needed which overcomes the shortcomings of such prior developments, particularly facilitating implantation, providing effective distributive forces and multi-axial support within target fracture sites, and imparting significant strength for suitable support as well as reduction in propensity for disengagement with a target bone over time.

### SUMMARY OF THE DISCLOSURE

The disclosed thread form, whether in terms of medical/surgical/osteopathic use or other type of industrial purpose, herein may be utilized for introduction within any number of bones and bone tissues within a human (or other animal body), particularly or ostensibly to facilitate and potentially accelerate bone (or other tissue) healing internally, when properly constructed for such an end use. Additionally, in terms of industrial usage, the same type of thread, albeit provided in proper size and length for such issues, may be utilized for introduction within any such industrial activity, as well. As such, as it concerns medical uses, without limitation, such a disclosed thread form may be applied to a fixation device and introduced within bones such as the femur, the patella, the tibia, the fibula, the radius, the ulna, the ankle, the foot, the wrist, the clavicle, the pelvis, the scapula, the vertabrae, the cranium, and the like. Basically, the ability to implant such a disclosed fixation apparatus is possible at most locations within the human body.

Accordingly, encompassed within this disclosure is an osteopathic screw apparatus, wherein said screw apparatus comprises a cylindrical base shaft having a top head component, a distal end component, and a thread configuration component helically wound around at least a part of said cylindrical base shaft, wherein said thread configuration comprises a uniformly shaped and spaced structure exhibiting an angled extension along the entirety of said thread configuration having repeated helical configuration along said cylindrical base shaft having a top edge and a bottom edge, wherein each said angled extension exhibits a distance of 0.25 mm to any distance with relative proportion as required for optimal results between the bottom edge of any helical extension to the bottom edge of a helical extension directly above, wherein each said bottom edge and top edge together are a perpendicularly disposed post from said cylindrical base shaft such that said bottom edge is disposed from 25-75% of the height of the top edge wherein said perpendicularly disposed post extends from said cylindrical base from 25-75% of the thread height in relation to both said top and bottom edges to a second angular portion of both said top and bottom edges, wherein said second angular portion of both said top and bottom edge is configured and disposed at a uniform angle of from 110 to 155 degrees, wherein said top edge leads to a point from 25-75% the total height of the thread at the perpendicular top edge post, and wherein said bottom edge leads to a point present at a distance of from 25 to 75% from the point along a straight line from said bottom edge second angular point, wherein such a configuration allows said top edge to end at a point that is closer to said cylindrical base shaft than that of said top edge, thereby creating a sharp taper at such a location. Furthermore, such an osteopathic screw apparatus may exhibit/include a cannula within said cylindrical base shaft as well as be non-cannulated.

Provided within this disclosure is a unique thread form, herein described in particularity as an osteopathic screw (although the thread disclosed herein may be employed, as noted above, in any type of screw structure) exhibiting a specific thread structure for increased load bearing, distributive forces, and multi-axial support within a target bone. The thread structure disclosed herein imparts further beneficial properties in relation to bone implantation, particularly, without limitation, increased pullout strength to reduce the propensity of unwanted disassociation of the screw from the target location, as well as a reduce propensity for bone stripping due to screw loosening and/or microfracturing of such bones from within the screw channel due to improved torque resistance thereof, and further variable loading at different angles of implantation. Such a screw is preferably, though not necessarily, cannulated to allow for effective bone introduction (as well as possible fluid/medicament introduction therein a target bone if necessary) and includes a washer component to effectuate a more secure implantation within a target bone, as well.

The threaded structure of the disclosed osteopathic screw includes helical structures wound around a cylindrical base with the thread configuration exhibiting an extended and angled portion thereof as it continuously winds around such a base. Such an extended thread exhibits such a repeated helical configuration with either a repeated distance of from 0.5 mm to any number of distance with relative proportion as required for optimal results or with a variable but coordinated between the bottom edge of any helical extension to the bottom edge of a helical extension directly above (and thus in repetition for each helical extension in the same manner). Such a distance may be 1 millimeter as a preferable measurement but could range to any suitable size in relation to the screw, wire, or pin itself. The helical extensions thus include a perpendicularly disposed post at any position along the cylindrical base structure that has the above-noted bottom edge. This perpendicularly disposed post extends from the cylindrical base from 25 to 75% of the total height, preferably from 42 to 46%, most preferably 44%, for both the top and bottom edges thereof. From that point, a second angular portion of both top and bottom edges is present with both edges configured and disposed at angles of from 110 to 155 degrees, preferably from 132 to 133 degrees, with the top edge leading to a point from 25 to 75% of the height above the perpendicular top edge post, preferably at a height ˜60%, and with the bottom edge leading to a point present at a distance approximately 50-60% total height from the point of the angular portion (in an axial line therefrom, thus with the angled bottom edge potion endpoint ending at such a distance in the plane of the bottom edge post). Such a configuration allows for the top edge to end at a point that is closer to the cylindrical base than that of the top peak edge, creating a sharp taper at such a location, again as a continuous helical structure along the entirety of each helical repeat. Such a threading configuration is thus present along the entirety of the screw (with the bottom thereof tapered in a typical fashion to allow for an initial insertion and screw motion for implantation within a target bone) or only for a partial length of a base screw (for example, the top portion of the cylindrical screw base may be free from any helical thread configuration, and the noted disclosed threaded configuration introduced along the body of the screw leading downward to the tapered end. In this manner, the screw may be provided in a manner that is conducive to different types of implant sites, allowing a user the ability to determine the most effective type of unique threaded osteopathic screw as disclosed to be utilized as needed.

The full length of the disclosed screw with such a unique threaded configuration may be of any length associated with bone fracture sites within a body (human or other animal). In medical application these lengths frequently range from 5-200 millimeters, but can vary without limitation. The actual length along the screw base (body) may be anywhere from 5 up to 200 mm, as well. Wires and pins will commonly have variance of 5 to more than 300 millimeters. The cannula structure of the disclosed screw may be of any bore associated with such osteopathic considerations, as well, that relates to the structural measurements noted above.

As noted above, this same screw thread configuration aids not only in terms of medical/surgical/osteopathic end uses, but also as it concerns 25 mm to any number of distance with relative proportion as required for optimal results to a plethora of different industrial purposes. Thus, again, as noted above, such thread descriptions are not intended and thus should be well understood to the ordinarily skilled artisan within such an area to be limited not just to medical uses, but myriad industrial purposes, as well.

In accordance with an aspect of this disclosure, a screw (such as, without limitation, a medical screw) is provided that comprises a head for receiving a screw-driving device and a cylindrical shaft extending from the head. The shaft generally includes respective helical threads around the cylindrical shaft with each thread exhibiting the aforenoted angled extension upward toward the location of the head itself. Such angled extensions (again, as defined above) are oftentimes basically in parallel disposition both in terms of the initial extension from the screw base (cylindrical shaft) as well as in terms of the angled extension, further exhibiting the same length of such an angled extension along the entirety of the thread portion of the overall screw. The cylindrical shaft is again preferably provided as a cannula structure, thereby requiring a shaft body that is sufficiently strong to retain the thread configuration and remain dimensionally stable when utilized and thus when implanted within a bone. In other words, the cylindrical shaft, when present in cannula form, must withstand the pressures of bone implantation as well as the forces applied during actual machining (manufacturing) of the specific angled extension structure of the thread configuration thereof.

Again, as noted previously, the importance of the angled extension is vital to the overall utilization of the disclosed medical screw, wire, or fixation pin for bone implantation (or other like medical purpose, not to mention, again, as alluded to above, any industrial uses that involve the necessity for strength and torque resistance). The angled extensions, being uniform, again, along the entirety of its presence on a cylindrical base shaft, provide a functional fixation apparatus that introduces easily within a subject bone and, significantly, provides a distribution of multi-axial forces within such a subject bone that allows for heretofore increased levels of fixation as well as a reduced propensity for disengagement upon utilization for an appreciable amount of time. These increases in force distribution within a target bone accords more reliable support with much reduced potential for microfracture thereof during long-term utilization, a much desirable result.

Additionally, there is possible the inclusion of a pressure-applied washer over the cannula head portion of the disclosed screw. Such a washer accords a number of benefits, primarily, perhaps, the ability to provide an attachable adapter for facilitation of screw removal from a target bone. Ostensibly, in one aspect, potentially, of this disclosure, such a pressure-applied washer may exhibit a shape that allows for interface with a suitable tool that engages properly for a user to unscrew the disclosed screw apparatus on demand. Additionally, such a washer may cover the cannula opening to prevent unwanted materials from entering such a hollow tube component of the disclosed screw, as well. Such a washer may further accord a means to attach a further implement or device to the disclosed screw if desired, as well.

The disclosed screw, as well as the washer noted above, may be manufactured from any number of materials, but, certainly, in terms of proper medical utilization thereof, certain metal structures are most preferred. These include, without limitation, but, again, potentially preferred, any type of bio-compatible material, for example, titanium alloy, stainless steel, class six implant grade plastic or a material made from bioabsorbables, such as polyglycolic acid, polyoxalic acid, and the like.

In terms of actual manufacturing of such a unique screw apparatus, the angled extensions from the cylindrical screw shaft body are not a simple structure to obtain. In particularly, again, uniform angled extensions (with the initial helical thread extending from the cylindrical screw shaft body at a certain angle and a certain length followed by the upward extension exhibiting an angle within a certain range and an extension length within a range, as well) are necessary to effectuate and implement the structural requirements and maximum force distribution capability and efficiency thereof upon bone implantation. The ability to produce such a specific structure requires significant machining adjustments with an initial base cylindrical structure (hollow in configuration potentially, as well, for the cannula structure thereof) that is thick enough to provide the capability of creating the angled extension within the thread portion thereof. Placing such a cylindrical base shaft on a spindle, the machining procedure may be followed to carefully maneuver the device to remove materials uniformly to retain the necessary thread configuration with the multiple and continuous helical thread base with the angled extensions included. Additionally, if desired, such a screw may be manufactured through the utilization of three-dimensional (3D) printing.

In accordance with another aspect of the present disclosure, the medical screw may be configured with a variety of different head types and shapes.

As alluded to above, the disclosed medical screw can exhibit any length, and the diameters of the shaft, the distance between the threads and thus the angled extension, may differ for different types and sizes of the bone in which the screw is to be implanted and/or attached.

Such a screw length may be, again, from 5 up to 200 millimeters, for example, and the diameter, for instance, may be from 2.0 to 8.0 millimeters (potentially larger). As it concerns possible industrial end uses, the length may range anywhere from 20 millimeters to 500 centimeters in length, with the diameter ranging anywhere from 10 to 100 centimeters. Again, the importance of the structure of the disclosed screw(s) resides in the described thread configuration(s) to impart the desired strength and torque resistance for optimal performance.

Additionally, and again as alluded to above, the disclosed medical screw comprises a distal guiding tip (tapered, at least, to a distance that is narrower than the screw head) that aids in the insertion of the screw into a pre-tapped/pre-drilled implantation site in target bone tissue.

The present disclosure thus further pertains to an interloading mechanism for an implant anchor, incorporating a novel external thread geometry with a triangular ullage space configuration. This innovative design ensures immediate interlocking of the implant anchor with the receiving bone cavity upon insertion. The advantages of this design include reduced healing time, streamlined procedural requirements, cost reduction, and minimized patient discomfort. Additionally, the VAL (Variable Angle Loading) thread configuration improves the anchor's resistance to shear forces encountered during occlusal activities, thereby enhancing the overall strength and stability of the implant site. Collectively, these features contribute to the long-term strength and durability of the implant.

The variable thread disclosed herein may be used on a variety of materials to generate less motion and potentially lessen product failure due to the design. Such a thread may be present or added to a material to create an implantable or securing device, particularly characterized by an interloading thread helically rotating around a longitudinal axis, such that the superior thread contact surface that forms an acute angle with both superior surfaces less than 90 degrees, as well as an interloading inferior flank that has approximately a 90% angle from the driving axis and a second angle that extends superior and radially with superior contact surfaces. In this manner, load forces may be multidirectional. Where load forces are multi-axial and/or multi-directional, or not unidirectional and axial, failure can occur. To prevent pullout from the from axial, side, or variable angle loading, the disclosed thread design incorporates a specific configuration of the contact surfaces, as described in greater detail below.

Of particular importance, perhaps, is the manner of which the entirety of such a fixation screw is designed for embedding into bone. In one embodiment, for instance, there is a screw body having a main structure featuring a distal end intended for penetration into the subject bone and stem end located opposite the distal end for external control thereof. Additionally, then, such a fixation screw will also include the disclosed thread structure herein, notably a helical thread circumscribing the screw body, extending between the stem end and the distal end, and spaced from the central longitudinal axis of the screw body, extending partially or fully. Such a thread exhibits an “L” shape with a triangular ullage when sectioned along a vertical plane intersecting the longitudinal axis and further exhibits a specific type of thread geometry. Such geometry encompasses the “L” shape with triangular ullage, noted above, including first side that defines a pair of contact surfaces, second side that defines a pair of inferior surfaces, and wherein both the contact surfaces and the inferior surfaces are integral to the “L” shape with triangular ullage. Such an overall configuration enhances the anchor screw's engagement with the bone, optimizing stability and resistance during use.

Furthermore, in terms of such contact surfaces, at least one of the first and second inferior surfaces defines an interlocking flank surface, whereas the other of the first and second inferior surfaces defines an inferior relief surface. Additionally, the interlocking flank surface is positioned closer to the longitudinal axis than the inferior relief surface. Such contact surfaces face the stem end and form a triangle ullage at their intersection. As well, the inferior surfaces also forms an “L” shape extension from the base and extends radially and superior at their intersection, such that the inferior surfaces face the distal end of the screw.

It is believed that the screw geometry is effective due to the thread geometry and triangle ullage shape, ostensibly due to the unique thread form inserts with the initial thread turns having less constraint and begins to tighten within the pilot hole as the thread form performs its action as intended of torquing within the bone without any delamination of bone.

It was further realized that the disclosed variable thread loading herein disclosed exhibits (within a screw including such thread) peak performance during the initial insertion within a subject bone. In essence, it was realized that testing during the prototype phase demonstrated that the first insertion Torque was just as great (slight decrease) for the multiple testing reps with the same screw. After the fourth test, for example, the screw edge became blunted and the Torque started to decrease. After 4 torque tests with same (simpliar) results during the prototype phase the screw was reviewed on a 3D MicroVue vision system and after the 4th insertion and torque test, the screw threads appeared blunted. Such a result shows the effectiveness of the thread disclosed herein and that, even upon strong and high-torque insertion, such threads remain as needed to ensure penetration and stability within subject bone(s).

Such screw geometry was created by using special/unique custom tooling made from Tool Steel, and/or Carbide. Such a manufacturing step was accomplished by precision programming using CNC machinery while allowing the Tool steel custom tooling to cut the threads in a manner that reduces friction and the end result is a very sharp cutting screw edge at the peak of the thread crest. The manufacturing process was proven out through the utilization of CNC Machining parameters within the CNC program, allowing for repeatability for short and long runs of screws, wires, pins, etc. (of all different sizes). Additionally, such manufacturing was undertaken using medical grade materials with FDA calibrated CNC.

The aforementioned cannulation (as an option) within the disclosed screw herein was undertaken, as well, as precision grade using a calibrated Gauge pin for inspection. Such an inspection process for the disclosed variable thread loading complex geometry screw requires an overlay thereof to ensure that the geometry for all sizes meet necessary thread design requirements, as well.

These and other aspects of the present disclosure will become more apparent to those skilled in the art from the following nonlimiting detailed description of preferred embodiments of the invention taken with reference to the accompanying figures and other medical screws.

### BRIEF DESCRIPTION OF THE DRAWINGS

The disclosed surgical/medical/osteopathic screw (in various embodiments) will hereafter be described in conjunction with the appended drawing figures, wherein like designations denote like elements.

FIG. 1 is a side cross-sectional view of a possible embodiment of the variable thread for a screw within the disclosure herein.

FIG. 2 is a side perspective view of the thread embodiment of FIG. 1 showing general measurements of a screw that in this example would be intended to go with a 7 mm screw that has a 5 mm core diameter.

FIG. 3 is a side cross-sectional view of an embodiment of the disclosure that shows the helical pattern in an enhanced view of the thread.

FIG. 3A is a side cross-sectional view of a larger portion of a screw with the helical pattern of FIG. 3.

FIG. 4 is a side view of a screw implant device in accordance with an embodiment of the disclosure.

### DETAILED DESCRIPTION OF THE DRAWINGS AND PREFERRED EMBODIMENTS OF THE DISCLOSURE

The following description is of exemplary medical screws involving the Variable Angle Loading Thread of this disclosure and is not intended to limit the scope, applicability or configuration of the disclosure in any way. Rather, the following description provides a convenient illustration for implementing exemplary embodiments of the disclosure. Various changes to the described embodiments may be made in the function and arrangement of the elements described without departing from the scope of the disclosure as set forth in the appended claims.

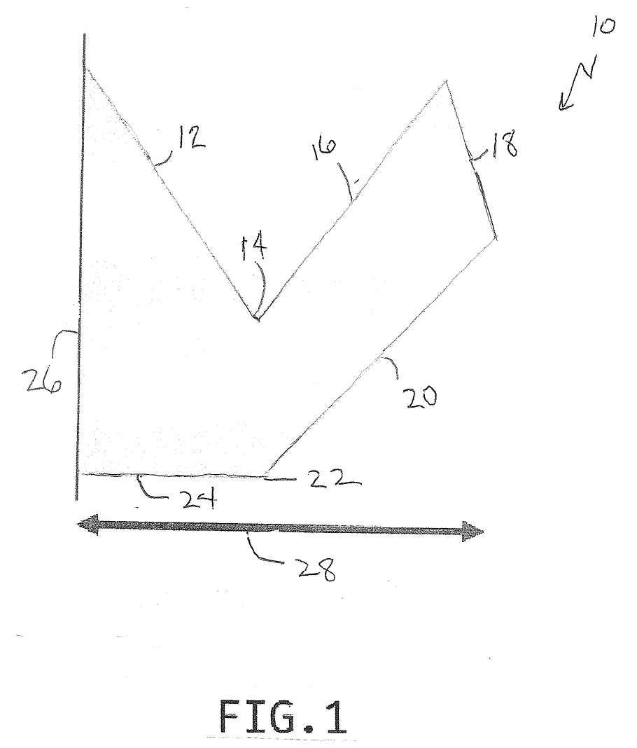

Referring to FIG. 1, a variable thread 10 is provided including a helical extension 18 from a base screw shaft 26 is provided as one potentially preferred embodiment of the disclosure. From the shaft 26 is an extension post 14 and 22 with both top edges 16 and 20, leading varying degrees of perpendicularity therefrom to a top edge. The loading of surfaces of 12 and 16 substantially aid in preventing toggle and pistoning movement of the helical end 18, thus keeping the thread position more stable. The extension 18 from the cylindrical base shaft 26 has a bottom edge 24 perpendicularly disposed from the cylindrical base shaft 26 such that said bottom edge is disposed from 5-25% below the top edge 12. The perpendicularly disposed bottom edge post 24 further extends from the cylindrical base 26 from 25 to 75% in relation to both a top edges extension 12 and a second angular portion thereof 16 (at an inflection point 14). Such a second angular portion 16 is configured and disposed at a uniform angle of from 110 to 155°, and leads to a point (FIG. 1. #17) from 75 to 95% of the total distance above the perpendicular top edge 16. The bottom edge 24 leads to a point 22 present at a distance of from 25 to 75% of the total distance from the point along a straight line from said second angular point 14, wherein such a configuration allows said top edge to end at a point that is closer to said cylindrical base shaft than that of said top peak edge, thereby creating a sharp taper 18 at such a location. In this manner, screw loading (including such a thread 10) would primarily occur during insertion but possibly increased with micromotion after insertion. The external thread crest (taper 18) is defined helically around the circumference of the core. As well, the external thread 10 is characterized by two superior contact surfaces 12 and 16, and inferior side relief 20, and a interloading inferior flank 24. The interloading flank 24 forms an angle that is perpendicular to the axis of rotation often characterized in relation to the minor diameter forming an “L” shape from the core of the screw (shaft 26).

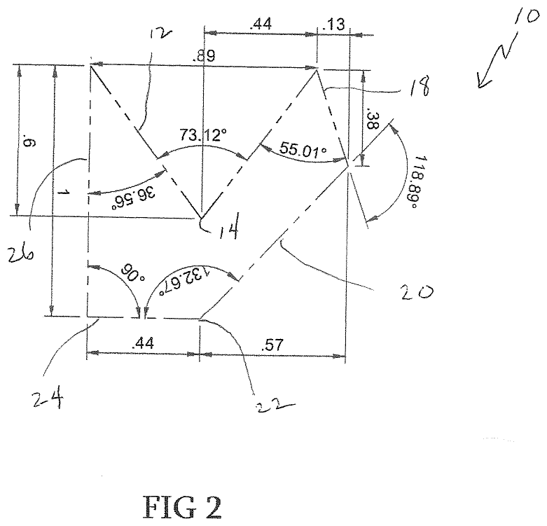

FIG. 2 shows a cross-sectional side view of the same thread 10 of FIG. 1, with particular measurements denoted in relation to a specific size screw (7 mm) with a 5 mm core. Such measurements are provided for each repetitive thread 10.

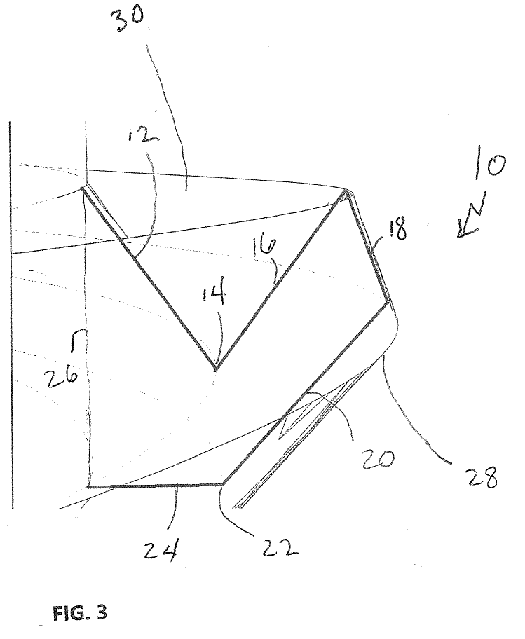

FIGS. 3 and 3A display a transparent cross-sectional view of a screw including the thread 10 of FIG. 1. Additionally, in relation to the screw itself, there shown an upper edge 30 and a lower edge 28 in relation to the thread structures as defined above. The same edges 28, 30 are depicted in larger size in FIG. 3A, showing a number of repetitive threads 10 along the shaft 26 of the screw to permit the capabilities thereof described within this disclosure, particularly, though not necessarily, as it concerns the utilization for bone penetration and stability.

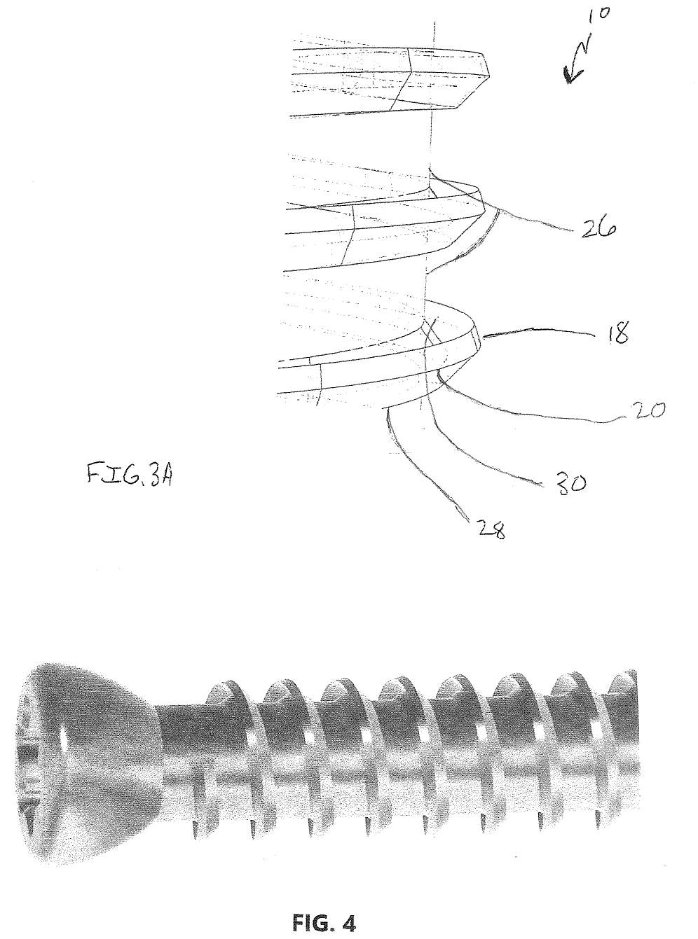

FIG. 4 provides a partial view of a screw including the thread of FIGS. 1 and 3, at least, and a head portion with a cannula therein. Such a screw of FIG. 4 thus provides a view of an actual screw implement/device incorporating the disclosed variable thread described herein for utilization, at least, within a surgical/medical procedure.

Such a thread on a screw, wire, pin or similar fixation device as described in relation to FIGS. 1 and 2, above, again as potentially preferred embodiments of this disclosure, may further be associated with and fitted with a washer partially applied (with pressure) to the screw head portion. This covers the cannula shaft as needed, thus allowing for introduction of an implement within such a cannula and covering/sealing thereof with the washer if preferred. Such a cannula and thus washer may exhibit complementary shapes, such as circular, spherical, square, hexagonal, octagonal, and the like (hexagonal potentially preferred). Such a washer may thus also allow for an adapter to apply over the screw head if desired by the user. As noted in FIG. 4, for instance, the screw head has an opening therein for any such cannulating purposes.

Although the subject disclosure has been described herein in conjunction with the appended drawing Figures, it will be appreciated that the scope of the disclosure is not so limited. Various modifications in the arrangement of the components discussed and the steps described herein for using the subject device may be made without departing from the disclosure as set forth in the appended claims.

What we claim is:

1. An osteopathic screw apparatus, wherein said screw apparatus comprises a cylindrical base shaft having a top head component, a distal end component, and a thread configuration component helically wound around at least a part of said cylindrical base shaft, wherein said thread configuration comprises a uniformly shaped and spaced structure exhibiting an angled extension along the entirety of said thread configuration having repeated helical configuration along said cylindrical base shaft having a top edge and a bottom edge, wherein each said angled extension exhibits a distance of from 0.25 mm to any number of distance with relative proportion as required for optimal results between the bottom edge of any helical extension to the bottom edge of a helical extension directly above, wherein each said bottom edge and top edge together are a perpendicularly disposed post from said cylindrical base shaft such that said bottom edge is disposed from 5-25% below said top edge, wherein said perpendicularly disposed post extends from said cylindrical base from 25 to 75% in relation to both said top and bottom edges to a second angular portion of both said top and bottom edges, wherein said second angular portion of both said top and bottom edge is configured and disposed at a uniform angle of from 110 to 155°, wherein said top edge leads to a point from 75 to 95% of the total mm above the perpendicular top edge post, and wherein said bottom edge leads to a point present at a distance of from 25 to 75% of the total mm distance from the point along a straight line from said bottom edge second angular point, wherein such a configuration allows said top edge to end at a point that is closer to said cylindrical base shaft than that of said top peak edge, thereby creating a sharp taper at such a location.

2. The osteopathic screw apparatus of claim 1 wherein said cylindrical base shaft is present as a cannula or non-cannula.
